(19) United States
(12) Patent Application Publication
(10) Pub. No.: US 2012/0263781 A1
(43) Pub. Date: Oct. 18, 2012

(54) METHODS AND COMPOSITIONS FOR TREATING RHINITIS

(75) Inventors: Michael B. Chancellor, Pittsburgh, PA (US); Jonathan H. Kaufman, Pittsburgh, PA (US)

(73) Assignee: Lipella Pharmaceuticals Inc.

(21) Appl. No.: 13/368,088

(22) Filed: Feb. 7, 2012

Related U.S. Application Data
(63) Continuation-in-part of application No. 12/651,075, filed on Dec. 31, 2009.
Application No. 12/651,075 is a continuation-in-part of application No. 11/546,025, filed on Oct. 11, 2006, now Pat. No. 8,110,217.
(60) Provisional application No. 60/725,402, filed on Oct. 11, 2005.
(60) Provisional application No. 60/311,868, filed on Aug. 13, 2001.

(51) Int. Cl. A61K 39/08; A61P 11/02; A61K 9/127

(52) U.S. Cl. 424/450; 424/239.1

(57) ABSTRACT

Improved efficacy in treatment of rhinitis with botulinum toxin is obtained using liposomal encapsulated botulinum formulations for administration of the botulinum toxin. The liposomes are typically administered in a physiologically acceptable carrier such as saline or phosphate buffered saline by instillation into the nasal passages.

### CROSS-REFERENCE TO RELATED APPLICATIONS

This application is a continuation-in-part of U.S. Ser. No. 12/651,075 filed Dec. 31, 2009, which claims priority to U.S. Ser. No. 11/546,025 filed Oct. 11, 2006, now U.S. Pat. No. 8,110,217, which claims priority to U.S. Ser. No. 60/701,431 filed Jul. 20, 2005 and U.S. Ser. No. 60/725,402 filed Oct. 11, 2005, and which is a divisional of U.S. Ser. No. 10/218,797 filed Aug. 13, 2002, now U.S. Pat. No. 7,063,860, which claims priority to U.S. Ser. No. 60/311,868 filed Aug. 13, 2001.

### FIELD OF THE INVENTION

The present invention is generally in the filed of compositions and methods for treatment of rhinitis with botulinum toxin.

### BACKGROUND OF THE INVENTION

Rhinitis is characterized by frequent sneezing, congestion and an itchy or runny nose. Rhinitis is one of the most common chronic conditions, affecting 10% to 30% of adults and up to 40% of children in the United States. There are two types of rhinitis: allergic and non-allergic.

Allergic rhinitis is caused by allergens in the air. The immune system identifies pollen as an invader, or allergen, and overreacts by producing antibodies called Immunoglobulin E (IgE). These antibodies travel to cells that release chemicals, causing an allergic reaction with symptoms such as sneezing, stuffiness, a runny nose, itching and post-nasal drip. People with this condition are prone to itchy, watery eyes (from allergic conjunctivitis), and they may be more sensitive to irritants such as smoke, perfume or cold, dry air. Rhinitis can contribute to other problems such as asthma, sinus or ear conditions or trouble sleeping. When allergic rhinitis is caused by outdoor allergens such as tree, grass and weed pollen, it is called seasonal allergic rhinitis (hay fever). Rhinitis can also occur year-round because of indoor allergens from pets, mold, dust mites and cockroach droppings. This is called perennial allergic rhinitis. An individual can have either seasonal or perennial allergic, or a combination of both.

Steps to manage the symptoms include avoiding the allergens, medications and/or allergy shots (immunotherapy). Some medications for allergic rhinitis are best used daily to control inflammation and prevent symptoms, while others are used only as needed to relieve symptoms. Nasal corticosteroid sprays can control inflammation and reduce all symptoms, including itching, sneezing, runny nose and stuffiness. Antihistamines in the form of pills or nasal sprays block histamine and may relieve itching, sneezing and runny nose, but they may not be as effective in reducing nasal stuffiness. Anti-leukotrienes can reduce all the symptoms. Decongestant pills or nasal sprays can be used as needed if nasal stuffiness is not relieved with other medications. Decongestant sprays should not be used for long periods of time because they can cause the congestion to return.

Allergy shots may be administered if the symptoms are constant, if medications are insufficient, or for long-tellu control of the allergies with less need for medications. This treatment involves receiving injections periodically, typically over a period of three to five years.

Non-allergic rhinitis (also known as intrinsic rhinitis) usually begins in adults and causes year-round symptoms, especially a runny nose and nasal stuffiness. Strong odors, pollution, smoke and other irritants may non-allergic rhinitis and symptoms thereof. Non-allergic rhinitis symptoms can also develop as side effects of medications, including some blood pressure medicines, oral contraceptives or medications used for erectile dysfunction. The most common form of this type of non-allergic rhinitis is caused by nasal decongestant sprays such as oxymetazoline, when used for long periods of time. This type of medication-induced rhinitis is also called rhinitis medicamentosa.

A typical treatment for an individual with nasal inflammation is nasal corticosteroid sprays. Ipratropium nasal spray can be administered to relieve a runny nose. Decongestant pills can be used as needed to relieve nasal inflammation.

All of the current treatments have disadvantages. In some cases, treatment requires frequent dosing with drugs that have systemic effects such as drowsiness or sleeplessness, dry mouth, and gastrointestinal distress. hi other cases, the treatment requires allergy shots. It is therefore desirable to have an alternative therapy for long term relief from the symptoms of rhinitis.

Several groups have investigated botulinum toxin as an alternative treatment for the symptoms of rhinitis. For example, Nowak, et al., Otolaryngol Pol. 65(2):103-5 (2011), described administration via injection botulinum toxin to the nasal turbinates in patients to diminish their symptoms of patients reporting symptoms stuffy nose, sneezing, and runny nose. They reported that all patients had an improvement, with the full effect of the toxin within 1-2 weeks after application and lasting 8-12 weeks. Rohrbach, et al., ORL J Otorhinolaryngol Relat Spec. 63(6):382-4 (2001) described the effect of the local application of botulinum toxin A on nasal hypersecretion in a female patient with intrinsic rhinitis. 20 units of botulinum toxin A (Botox) were inserted into each nostril using a small sponge in close contact with the lower and middle nasal turbinates. Nasal hypersecretion diminished clearly 5 days after the treatment. The rhinomanometric flow increased 2 weeks after the application. Sapci, et al. reported in Rhinology. 46(1):45-51 (2008) on the effects of botulinum on idiopathic rhinitis without eosinophilia. One of the most disturbing symptoms for patients within this disease group is nasal hypersecretion. Although many different treatments have been tried for hypersecretion, nasal topical drugs form the basis of any such therapy today. Ipratropium bromide (IB) is a drug of first choice in nasal hypersecretion therapy. It displays a parasympatholytic effect when topically administered and antagonizes acetylcholine transport in efferent parasympathetic nerves, thus decreasing submucosal gland secretion, which is the cause of hypersecretion. Botulinum toxin type A (BoNT-A) is among the alternative treatment choices that is increasingly used in symptomatic treatment of nasal hypersecretion. Sapci's study demonstrated that although IB and BoNT-A differ in terms of method of application, they display a similar degree and duration of efficiency in hypersecretion therapy.

In all reported cases, botulinum toxin was effective in decreasing some symptoms of rhinitis for a period of one to two weeks. However, results varied and in many cases were not better than alternative treatments.

It is therefore an object of the present invention to provide improved compositions and methods of administration of botulinum toxin to provide relief from one or more symptoms of rhinitis.

It is a further object of the present invention to provide compositions and methods of administering the compositions that provide relief from one or more symptoms of rhinitis that is effective for a prolonged period of time compared to current therapies.

### SUMMARY OF THE INVENTION

Formulations containing botulinum toxin (BoNT) encapsulated in liposomes and suspended in a pharmaceutically acceptable carrier are administered intranasally to treat one or more symptoms of rhinitis. The BoNT can be BoNT A-G, preferably BoNT A, C or E, more preferably BoNT A. The liposomes are preferably formed of phospholipids or sphingolipids. The molar ratio of a phospholipid to second lipid can range from about 5:1 to about 1:1. The formulations can be in the form of a liquid or gel, preferably a liquid administered as a suspension, aerosol or spray. The dosage formulation can be a single use or multiple use formulation. It is preferably provided in the form of a dry powder in combination with diluent for resuspension.

The formulations can be administered in a dose of BoNT from about 1 to about 100 units, preferably about 1 to about 25 units, for treatment. The formulations can treat allergic and non-allergic rhinitis and symptoms associated with rhinitis. Such symptoms include, but are not limited to, nasal congestion, sneezing, rhinorrhea, postnasal drip, nasal pain, sinus pain, headache, coughing, wheezing, itching, redness, thickened nasal mucosa, and nasal polyp. The foiniulations can be administered once daily, preferably once daily weekly.

### DETAILED DESCRIPTION OF THE INVENTION

### I. Formulations

Liposome encapsulation increases absorption of botulinum toxin after instillation. Liposome encapsulation also protects BoNT from degradation in vivo and allows unhindered absorption across the tissue from liposomes adhering to the tissue surface. Since BoNT is entrapped inside the liposomes, it is not vulnerable to dilution by physiological secretions and localized concentration of BoNT at the liposome surface can be high enough to hasten the entry of leached BoNT from liposomes adhering to the surface of nasal lumen.

Botulinum toxin is a large protein (molecular weight≈150 kDa) which does not diffuse through tissue easily to reach its target. The target protein for BoNT resides in a lipid environment. Liposomes can enhance the activity of metalloproteases such as BoNT by allowing more efficient delivery of the BoNT to the tissue.

A. Liposomes

Liposomes are spherical vesicles, composed of concentric phospholipid bilayers separated by aqueous compartments. Liposomes adhere to and create a molecular film on cellular surfaces. (Gregoriadis, et al., Int J Pharm 300, 125-30 2005; Gregoriadis and Ryman, Biochem J 124, 58P (1971)). The lipid vesicles comprise either one or several aqueous compartments delineated by either one (unilamellar) or several (multilamellar) phospholipid bilayers (Sapra, et al., Curr Drug Deliv 2, 369-81 (2005)). The success of liposomes in the clinic has been attributed in part to the nontoxic nature of the lipids used in their formulation. Both the lipid bilayer and the aqueous interior core of liposomes can serve the purpose of treatment. Liposomes have been well studied as carrier of toxins for enhancing their efficacy at lower doses (Alam, et al., Mol Cell Biochem 112, 97-107 1992; Chaim-Matyas, et al., Biotechnol Appl Biochem 17 (Pt 1), 31-6 1993; de Paiva and Dolly, FEBS Lett 277, 171-4 (1990); Freitas and Frezard, Toxicon 35, 91-100 (1997); Mandal and Lee, Biochim Biophys Acta 1563, 7-17 (2002)).

Liposomes have been widely studied as drug carriers for a variety of chemotherapeutic agents (thousands of scientific articles have been published on the subject) (see, e.g. Gregoriadis, N Engl J Med 295, 765-70 (1976); Gregoriadis, et al., Int J Pharm 300, 125-30 (2005)). Water-soluble anticancer substances such as doxorubicin can be protected inside the aqueous compartment(s) of liposomes delimited by the phospholipid bilayer(s), whereas fat-soluble substances such as amphotericin and capsaicin can be integrated into the phospholipid bilayer (Aboul-Fadi, Curr Med Chem 12, 2193-214 (2005); Tyagi, et al., J Urol 171, 483-9 (2004)). Topical and vitreous delivery of cyclosporine was drastically improved with liposomes (Lallemand, et al., Eur J Pharm Biopharm 56, 307-18 2003). Delivery of chemotherapeutic agents lead to improved pharmacokinetics and reduced toxicity profile (Gregoriadis, Trends Biotechnol 13, 527-37 (1995); Gregoriadis and Allison, FEBS Lett 45, 71-4 1974; Sapra, et al., Curr Drug Deliv 2, 369-81 (2005)). More than ten liposomal and lipid-based formulations have been approved by regulatory authorities and many liposomal drugs are in preclinical development or in clinical trials (Barnes, Expert Opin Pharmacother 7, 607-15 (2006); Minko, et al., Anticancer Agents Med Chem 6, 537-52 (2006)). Fraser, et al. (Urology, 2003; 61: 656-663) demonstrated that intravesical instillation of liposomes enhanced the barrier properties of dysfunctional tissue and partially reversed the high micturition frequency in a rat model of hyperactive bladder induced by breaching the uroepithelium with protamine sulfate and thereafter irritating the bladder with KCl. Tyagi et al. J Urol., 2004; 171; 483-489 reported that liposomes are a superior vehicle for the intravesical administration of capsaicin with less vehicle induced inflammation in comparison with 30% ethanol. Clinical studies have proven the efficacy of liposomes as a topical healing agent (Dausch, et al., Klin Monatsbl Augenheilkd 223, 974-83 (2006); Lee, et al., Klin Monatsbl Augenheilkd 221, 825-36 (2004)). Liposomes have also been used in ophthalmology to ameliorate keratitis, corneal transplant rejection, uveitis, endophthalmitis, and proliferative vitreoretinopathy (Ebrahim, et al., Surv Ophthalmol. 50(2):167-82 (2005); Li, et al., 2007). The safety data with respect to acute, subchronic, and chronic toxicity of liposomes has been assimilated from the vast clinical experience of using liposomes in the clinic for thousands of patients. The safe use of liposomes for the intended clinical route is also supported by its widespread use as a vehicle for anticancer drugs in patients.

### a. Lipids

The liposomes contain one or more lipids. The lipids can be neutral, anionic or cationic lipids at physiologic pH.

Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including, but limited to, 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids. In one embodiment, the liposomes contain a phosphaditylcholine (PC) head group, and preferably sphingomyelin. In a preferred embodiment, the liposomes contain DPPC. In a preferred embodiment, the liposomes contain a neutral lipid, preferably 1,2-dioleoylphosphatidylcholine (DOPC).

In one embodiment, the formulations contain non-cationic liposomes, preferably of sphingomyelin, and a pharmaceutically acceptable carrier. In a further embodiment, the liposomes include a sphingomyelin metabolite and at least one lipid. Sphingomyelin metabolites includes, for example and without limitation ceramide, sphingosine or sphingosine 1-phosphate.

The concentration of the sphingomyelin metabolites included in the lipids used to formulate the liposomes can range from about 0.1 mol % to about 10.0 mol %, preferably from about 2.0 mol % to about 5.0 mol %, and more preferbly can be in a concentration of about 1.0 mol %.

Suitable cationic lipids in the liposomes include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N-(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N,N,N′, N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids may be formed from a combination of more than one lipid, for example, a charged lipid may be combined with a lipid that is non-ionic or uncharged at physiological pH. Non-ionic lipids include, but are not limited to, cholesterol and DOPE (1,2-dioleolylglyceryl phosphatidylethanolamine), with cholesterol being most preferred. The molar ratio of a first phospholipid, such as 1,2-diacyl-glycero-3-phosphocholines, to second lipid can range from about 5:1 to about 1:1 or 3:1 to about 1:1, more preferably from about 1.5:1 to about 1:1, and most preferably, the molar ratio is about 1:1.

### b. Liposome Core

The liposomes typically have an aqueous core. The aqueous core can contain water or a mixture of water and alcohol. Suitable alcohols include, but are not limited to, methanol, ethanol, propanol (such as isopropanol), butanol (such as n-butanol, isobutanol, sec-butanol, tert-butanol), pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

### c. Ratio of BoNT to Lipid

The BoNT to lipid ratio (unit of BoNT per mg of lipid) can be controlled to regulate the efficiency of the BoNT. Suitable BoNT to lipid ratios include, but are not limited to, 1:1, 1:0.9, 1:0.8, 1:0.7, 1:0.6, 1:0.5, 1:0.4, 1:0.3, 1:0.2 or 1:0.1 (unit of BoNT per mg of lipid). In one embodiment, the BoNT to lipid ratio is 1:0.5.

B. Botulinum Toxin (BoNT) and Other Drugs for Instillation

Botulinum neurotoxin (BoNT) refers to botulinum serotypes A, B, C, D, E, F, G and all modified, substituted or fragment versions of these toxins that have a blocking effect on snare proteins. These include any substitution or modification of at least 1 amino acid of a naturally produced toxin or synthetically produced toxins. These modifications can be made with recombinant techniques. Also included are toxins with removal or substitution of the binding domain and/or translocation domain. Some of these variations of BoNT types A to G are discussed in U.S. Pat. No. 7,491,799 and by Bland et al. (Protein Expr Purif., 71(1):62-73 (2010)).

Botulinum toxin is produced by Clostridium botulinum and is regarded as the most potent biological toxin known (Smith & Chancellor, J Urol, 171: 2128 (2004)). BoNT has been used effectively to treat different conditions with muscular hypercontraction. BoNT-A is the most common clinically used botulinum toxin among seven immunologically distinct neurotoxins (types A to G). BoNT-A and BoNT-B have been used successfully for the treatment of spinal cord injured patients with neurogenic bladder hyperactivity using intradetrusor BoNT-A injection at multiple sites.

BoNT is known to exert effects by inhibiting acetylcholine (“ACh”) release at the neuromuscular junction as well as autonomic neurotransmission. After intramuscular injection of BoNT, temporary chemodenervation and muscle relaxation can be achieved in skeletal muscle as well as in smooth muscle (Chuang & Chancellor, J Ural. 176(6 Pt 1):2375-82 (2006)). Smith et al. (J Urol, 169: 1896 (2003)) found that BoNT injection into the rat proximal urethral sphincter caused marked decreases in labeled norepinephrine at high but not at low electrical field stimulation, indicating that BoNT inhibits norepinephrine release at autonomic nerve terminals.

In one embodiment, the BoNT can be BoNT A-G, preferably BoNT A, C or E, more preferably BoNT A.

The formulations or liposomes optionally contain one or more drugs in place of or in addition to BoNT. These may include antiinfectives such as drugs to treat infections caused by bacteria, fungus, or viruses, antihistamines, analgesics, anti-inflammatories, decongestants, mucolytics, or other drugs used to treat rhinitis or sinus conditions.

C. Carriers

The formulations contain a pharmaceutically acceptable carrier, preferably a pharmaceutically acceptable aqueous carrier suitable for use in a nasal spray device. Suitable carriers include, but are not limited to, water or aqueous solutions containing pharmaceutically acceptable salts, buffers, or mixtures thereof, for example saline or phosphate buffered saline (PBS).

The concentration of liposomal BoNT in the carrier can be varied. Suitable concentrations of liposomal BoNT in the carrier include, but are not limited to, 0.05 mg/ml to 10 mg/ml, preferably 0.05 mg/ml to 5 mg/ml, more preferably 0.05 mg/ml to 2.5 mg/ml.

### II. Methods of Manufacturing

A. Manufacturing of Liposomes

Methods of manufacturing liposomes are described in the literature cited above and are well known. In one embodiment, aqueous liposome suspensions are produced by microfluidization. However, the end product may be subject to a series of stability problems such as aggregation, fusion and phospholipid hydrolysis (Nounou, et al., Acta Pal Pharm 62, 381-91 (2005)).

The liposomal product must possess adequate chemical and physical stability before its clinical benefit can be realized (Torchilin, Adv Drug Deliv Rev 58, 1532-55 (2006)). In a preferred embodiment, dehydrated liposomes are prepared using a suitable method For example, in a preferred embodiment dehydrated liposomes formed from a homogenous dispersion of phospholipid in a tert-butyl alcohol (TBA)/water cosolvent system. The isotropic monophasic solution of liposomes is freeze dried to generate dehydrated liposomal powder in a sterile vial. The freeze drying step leaves empty lipid vesicles or dehydrated liposomes after removing both water and TBA from the vial. On addition of a physiologically acceptable carrier, such as physiological saline or PBS, the lyophilized product spontaneously forms a homogenous liposome preparation (Amselem, et al., J Pharm Sci 79, 1045-52 (1990); Ozturk, et al., Adv Exp Med Biol 553, 231-42 (2004)). Liposomes having low lipid concentrations are well-suited for this method. The ratio of lipid to TBA is an important factor affecting the size and the polydispersity of resulting liposome preparation.

B. Preparation for Liposomal BoNT

In one embodiment, liposomal BoNT is prepared by a dehydration-rehydration method. Formulation of potent bacterial toxins into liposomes requires a meticulous approach. BoNT can not be exposed to organic solvents that are generally used in manufacture of liposomes. In a preferred method, liposomes encapsulating BoNT are prepared using a thin film hydration method and the lipid dipalmitoyl phosphatidylcholine (DPPC). Briefly, a solution of DPPC in chloroform is first evaporated under a thin stream of nitrogen in a round bottom flask. The lipid film is dried overnight under vacuum. Dried lipids are then hydrated with aqueous BoNT solution or suspension.

After liposomes are prepared, the liposomes are hydrated with a solution of BoNT in water for injection having a suitable concentration, such as 50 units/ml, at 37° C. Then the mixture is incubated at the temperature of 37° C. for a suitable period of time to form oligolamellar hydration liposomes. For example, in a bench scale set up, the mixture was incubated using a water bath for 2 hours.

A cryoprotectant, such as mannitol, is added to the mixture at a suitable concentration, such as 0.5%, 1%, 2.5% or 5% cryoprotectant (w/v) prior to freezing. For example, mannitol may be added to the final mixture at a concentration of 0.5%, 1%, 2.5% or 5% mannitol (w/v), before freezing in acetone-dry ice bath. Mannitol acts as a cryoprotectant in the freeze-drying process. The frozen mixture is lyophilized for a suitable period of time, such as at −40° C. and 5 millibar overnight.

The lyophilized cake is then resuspended with saline to the desired final concentration of BoNT. The free BoNT is removed from entrapped BoNT by a suitable method, such as centrifugation at 12,000×g for 30 min using ultracentrifuge. After washing, the precipitates are again resuspended in saline, PBS, or another pharmaceutically acceptable carrier.

The formulations can be stored as liquid, gels and solids. In one embodiment, the formulations are frozen or refrigerated during storage to extend shelf-life. In one embodiment, the liposomes are provided in the form of a dry, powder containing dehydrated BoNT encapsulated liposomes. For example, the BoNT encapsulated liposomes can be provided in a dry (e.g. freeze-dried) form, and be reconstituted with an aqueous solution immediately prior to administration. Shortly, for example within 2 hours, before use the dry powder is reconstituted in a pharmaceutically acceptable aqueous carrier. The BoNT encapsulated liposomes can be hydrated by dispersing the liposomes in an aqueous solution with vigorous mixing.

III. Treatment of Rhinitis or Symptoms Thereof with the Liposomal BoNT Formulations

The formulations containing liposomal BoNT and the carrier can be administered to the nasal passages by spraying liquid in to or administering gel-like material into the nasal passages. The formulations containing liposomal BoNT can be delivered in the form of an aerosol. In one embodiment, the formulations containing the liposomal BoNT are propelled into the nasal passages using a pressure source. In one embodiment, jet injection can be used to propel liquid or particles at great speed. The depth of penetration of the liposomal BoNT depends on the design of the nozzle and these parameters are known in the art. Following administration to the nasal passages, the liposomal BoNT deposits onto the nasal cavity walls.

Liposome encapsulation of BoNT solves the problem of poor absorption of BoNT after instillation demonstrated with prior art formulations of unencapsulated BoNT. BoNT entrapped inside the liposomes is not vulnerable to dilution by physiological secretions; and localized concentration of BoNT at the liposome surface is high enough to increase the rate of the passive diffusion of leached BoNT from liposomes adherent on the nasal surface through the nasal surface. The lipid barrier of liposomes can also prevent the access of proteases and proteinases in physiological secretions from cleaving the BoNT before it is absorbed by the nasal tissues.

One advantage with liposomal BoNT delivery is the ability to decrease dosage compared to the dosage required when administering a formulation of unencapsulated BoNT, while achieving the same therapeutic effect. The liposomes enhance the delivery of BoNT resulting in the effectiveness of lower dosages. The liposomal BoNT delivery also increases the effectiveness of treatment for a specified dosage of BoNT. For example, liposomal delivery of BoNT produces a more effective treatment compared to injection or application of only BoNT, such as that described in Rohrbach, et al., in ORL Otorhinolaryngol Relat Spec. 63(6):382-4 (2001), wherein 20 units of botulinum toxin A (Botox) was inserted into each nostril using a small sponge in close contact with the lower and middle turbinates which cleared nasal hypersecretion five days after administration.

Improved efficacy in treatment of rhinitis with botulinum toxin is obtained using liposomal encapsulated botulinum formulations for administration of the botulinum toxin. The liposomes are typically administered in a physiologically acceptable carrier such as saline or phosphate buffered saline by instillation, spraying, aerosolization, or dry powder into the nasal passages.

In one embodiment, the dose of BoNT is from about 1 to about 100 units. In a more preferred embodiment, the dose of BoNT is from about 1 to about 50 units. In a more preferred embodiment the dose of BoNT is from about 1 to about 25 units. In a most preferred embodiment the dose of BoNT is from about 1 to about 10 units.

Different size dosage units may be used. A dosage unit containing a dry powder of the dehydrated liposomal BoNT can be reconstituted in a container with a pharmaceutically acceptable carrier, preferably a pharmaceutically acceptable aqueous carrier. Suitable amounts include, but are not limited to, 0.1-1 mg, 1-3 mg, 3-10 mg, 10-20 mg and 20-50 mg. Suitable concentrations include, but are not limited to, 0.05 mg/ml to 10 mg/ml, preferably 0.05 mg/ml to 5 mg/ml, more preferably 0.05 mg/ml to 2.5 mg/ml.

The volume of formulation containing the liposome-BoNT is important in the efficacy of delivery. Routine experimentation can be used to determine the delivery volume.

The dosage formulation can be a single dose formulation or a multiple dose formulation. The dosage formulation can come in a single container with a divider between the carrier and the dry powder. The divider can be removed and the dosage formulation can be created by mixing the carrier and dry powder. The dosage formulation can be stored to increase the shelf life of the formulation, for example in a freezer or refrigerator.

A single administration can be effective for more than one week, preferably more than two weeks, more preferably more than three weeks following administration.

The formulation can be administered as required to provide effective relief from rhinitis or symptoms associated with rhinitis. For example, the formulation can be administered once or twice daily, once every two days, once every three days, once weekly, once every two weeks or once monthly over a predetermined period of time. The formulation containing liposomal BoNT is administered to a patient with rhinitis in a sufficient dose to alleviate rhinitis or one or more symptoms of rhinitis. Any type of rhinitis, such as infectious rhinitis, allergic rhinitis or intrinsic rhinitis, may be treated using the formulations described herein. Symptoms that may be alleviated following administration of the formulation include nasal congestion, sneezing, rhinorrhea, postnasal drip, nasal pain, sinus pain, headache, coughing, wheezing, itching, redness, thickened nasal mucosa, and nasal polyp.

Other symptoms and diseases that can be treated by the administration of formulations containing liposomal BoNT to the nasal passages, or to the nerves innervating these structures include sinusitis, asthma, COPD (bronchitis and emphysema), migraine headaches, impaired cerebral blood flow, headaches and sleep breathing disorders.

The mucosa may thin following the administration of liposomal BoNT, which increases mucus drainage and allows greater airflow within the patient's lungs. BoNT also causes changes in airway reflexes, thereby improving sleep disordered breathing, asthma, and COPD. BoNT also causes changes in vasomotor reflexes and tone, thereby improving cerebral circulation and tone.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A pharmaceutical composition comprising a lipid vehicle and a botulinum toxin, wherein the pharmaceutical composition is suitable for nasal administration.

2. The pharmaceutical composition of claim 1, wherein the pharmaceutical composition is a suspension, gel or dry powder.

3. The pharmaceutical composition of claim 1, wherein the botulinum toxin is selected from the group consisting of botulinum toxin A, botulinum toxin B, botulinum toxin C, tobulinum toxin D, botulinum toxin E, botulinum toxin F and botulinum toxin G.

4. The pharmaceutical composition of claim 1, wherein the lipid vehicle is selected from the group consisting of a micelle, an emulsion and a liposome.

5. The pharmaceutical composition of claim 1, wherein the lipid vehicle is a liposome.

6. The pharmaceutical composition of claim 5, wherein the liposome comprises a lipid selected from the group consisting of a phospholipid, a glycolipid, a sphingolipid, sphingophospholipid, and a sphingoglycolipid.

7. The pharmaceutical composition of claim 6, wherein the lipid is selected from the group consisting of phosphatidylcholine, phosphatidylethanolamine, phosphatidylserine, phosphatidylinositol, phosphatidylglycerol, cardiolipin, glycolipids, sphingomyelin, sphingosine I-phosphate, ceramide galactopyranoside, gangliosides, cerebroside, cholesterol, 1,2-distearoylsn-glycero-3-phosphocholine, 1,2-dioleoylphosphatidylcholine and combinations thereof.

8. The method of claim 5, wherein the liposome comprises one or more lipids, and wherein the ratio of botulinum toxin to lipid ranges from 1:1 to 1:0.1.

9. A method for treating rhinitis comprising administering to an individual in need thereof an effective amount to alleviate one or more symptoms of rhinitis of a pharmaceutical composition comprising a lipid vehicle and a botulinum toxin, wherein the pharmaceutical composition is suitable for nasal administration.

10. The method of claim 9, wherein the pharmaceutical composition is a suspension, gel or dry powder.

11. The method of claim 9, wherein the botulinum toxin is selected from the group consisting of botulinum toxin A, botulinum toxin B, botulinum toxin C, tobulinum toxin D, botulinum toxin E, botulinum toxin F and botulinum toxin G.

12. The method of claim 9, wherein the lipid vehicle is selected from the group consisting of a micelle, an emulsion and a liposome.

13. The method of claim 9, wherein the lipid vehicle is a liposome.

14. The method of claim 13, wherein the liposome comprises a lipid from the group consisting of selected from a phospholipid, a glycolipid, a sphingolipid, sphingophospholipid, and a sphingoglycolipid.

15. The method of claim 14, wherein the lipid is selected from the group consisting of phosphatidylcholine, phosphatidylethanolamine, phosphatidylserine, phosphatidylinositol, phosphatidylglycerol, cardiolipin, glycolipids, sphingomyelin, sphingosine I-phosphate, ceramide galactopyranoside, gangliosides, cerebroside, cholesterol, 1,2-distearoylsn-glycero-3-phosphocholine, 1,2-dioleoylphosphatidylcholine and combinations thereof.

16. The method of claim 13, wherein the liposome comprises one or more lipids, and wherein the ratio of botulinum toxin to lipid ranges from 1:1 to 1:0.1.

17. The method of claim 9, wherein the symptoms are selected from the group consisting of nasal congestion, sneezing, rhinorrhea, postnasal drip, nasal pain, sinus pain, headache, coughing, wheezing, itching, redness, thickened nasal mucosa, and nasal polyp.

18. The method of claim 9, wherein the dose of the botulinum toxin is from about 1 to about 25 units.

19. The method of claim 9, wherein the formulation is administered to the nasal passages by spraying or aerosolization.

20. The method of claim 9 further comprising administering with the composition agents selected from the group consisting of antiinfectives, antihistamines, analgesics, anti-inflammatories, decongestants, anti-mucolytics, and other drugs used to treat rhinitis or sinus conditions

21. The method of claim 9, wherein the formulation provides effective alleviation after administration for least 1 week, preferably 2 weeks, more preferably 3 weeks.

22. A dosage formulation of a dry powder which is reconstituted with a pharmaceutically acceptable aqueous carrier for treating rhinitis, wherein the dosage formulation comprises
(a) a container of between 0.1 and 1 mg, between greater than 1 and 3 mg, between greater than 3 and 10 mg and between greater than 10 and 20 mg of dry powdered liposomes having botulinum toxin encapsulated therein, and
(b) a container comprising a pharmaceutically acceptable diluent for the liposomes,
wherein, upon reconstitution of the dry powder in the container with the pharmaceutically acceptable diluent suitable for spraying, a liposomal-botulinum mass concentration between 0.05 mg/ml to 10 mg/ml in solution is formed.
